(19) United States
(12) Patent Application Publication
(10) Pub. No.: US 2023/0043194 A1
(43) Pub. Date: Feb. 9, 2023

(54) LAMBERTIAN GAS CAVITIES ILLUMINATED BY MID-INFRARED LIGHT-EMITTING DIODES

(71) Applicant: TERAHERTZ DEVICE CORPORATION, Salt Lake City, UT (US)

(72) Inventor: Mark S. Miller, Salt Lake City, UT (US)

(21) Appl. No.: 17/791,443

(22) PCT Filed: Jan. 7, 2021
(86) PCT No.: PCT/US2021/012504

§ 371 Date: Jul. 7, 2022

Related U.S. Application Data
(60) Provisional application No. 62/958,173, filed on Jan. 7, 2020.

(51) Int. Cl. G01N 21/3504; G02B 5/02; G01N 33/00

(52) U.S. Cl. CPC G01N 21/3504; G02B 5/021; G02B 5/0284; G01N 33/0027; G01N 2201/062

(57) ABSTRACT

The present disclosure describes a Lambertian gas-sensing system that may enable gas sensing in a compact form factor. The Lambertian gas-sensing system may include a hollow cavity, one or more light-emitting diode (LED) illuminators, one or more light-absorption detectors, and a gas exchange manifold. The hollow cavity may mechanically integrate the gas exchange manifold, the one or more LED illuminators, and one or more light-absorption detectors (such as one or more optical detectors). The gas exchange manifold may introduce gas into the hollow cavity and the one or more LED illuminators may emit light into the hollow cavity through one or more ports. The one or more light-absorption detectors may receive light from the hollow cavity through one or more ports.

### CROSS-REFERENCE TO RELATED APPLICATIONS

This application claims priority to and the benefit of U.S. Provisional Patent Application No. 62/958,173, filed on Jan. 7, 2020, which is hereby incorporated by reference in its entirety.

### BACKGROUND

### Background and Relevant Art

Optical light sources can be used to sense gases. An optical light source shines light through a gas mixture that may include a target gas species. Optical-mechanical components (such as light filters, mirrors, and gratings) are chosen and arranged in the light path to emphasize light absorption at the wavelengths characteristic of the target gas. The light-source wavelengths and the optical-mechanical sampling geometry may be selected to emphasize light absorption at the wavelengths characteristic of the target gas. The increases and decreases in detected light signal indicate the presence and concentration of the target gas.

### BRIEF SUMMARY

In accordance with one aspect of the present disclosure, a system for gas sensing is disclosed. The system includes a container. The container includes a hollow cavity, an optical port, a sensing port, an input port that allows gas to be injected into the hollow cavity, and an output port that allows the gas to leave the hollow cavity. The system further includes a mid-infrared light-emitting diode (LED) configured to emit light into the hollow cavity through the optical port. The system further includes a mid-infrared photodiode configured to receive the light from the hollow cavity through the sensing port.

The light emitted by the mid-infrared LED may have a wavelength of 3 μm to 20 μm.

The mid-infrared LED may be a high-brightness mid-infrared LED.

The hollow cavity may be a rectangular prism.

The hollow cavity may be spherical.

The container may include an internal surface surrounding the hollow cavity. The internal surface may be diffusively reflective. The internal surface may be reflective, have low absorption, and scatter the light in a Lambertian pattern. The internal surface may include aluminum.

The container may include plastic and have an internal surface surrounding the hollow cavity. The internal surface may include aluminum.

The container may include a first part and a second part. The first part may be detachable from the second part. The first part and the second part may fit together to form the hollow cavity.

In accordance with another aspect of the present disclosure, a method for gas sensing is disclosed. The method includes injecting gas into a hollow cavity of a container. The container includes an optical port and a sensing port. The method further includes emitting light from a mid-infrared light-emitting diode (LED) into the hollow cavity through the optical port. The method further includes receiving, through the sensing port, the light at a mid-infrared photodiode.

The method may further include allowing at least a portion of the gas to leave the hollow cavity.

The light emitted from the mid-infrared LED may have a wavelength of 3 μm to 20 μm.

The mid-infrared LED may be a high-brightness mid-infrared LED.

The hollow cavity may be a rectangular prism.

The hollow cavity may be spherical.

The container may include an internal surface surrounding the hollow cavity. The internal surface may be diffusively reflective. The internal surface may be reflective, have low absorption, and scatter the light in a Lambertian pattern. The internal surface may include aluminum.

The container may include plastic and an internal surface surrounding the hollow cavity. The internal surface may include aluminum.

The container may include a first part and a second part. The first part may be detachable from the second part. The first part and the second part may fit together to form the hollow cavity.

In accordance with another aspect of the present disclosure, a Lambertian gas cavity is disclose. The Lambertian gas cavity includes a diffusive light-storage cavity. The diffusive light-storage cavity stores isotropic light-field energy using diffusively reflective boundaries. The Lambertian gas cavity further includes a gas exchange manifold configured to supply a sample gas to the diffusive light-storage cavity. The Lambertian gas cavity further includes a high-efficiency LED illuminator configured to emit light into the diffusive light-storage cavity. The Lambertian gas cavity further includes a light absorption detector configured to receive light from the diffusive light-storage cavity.

The gas exchange manifold may actively supply the sample gas to the diffusive light-storage cavity. The gas exchange manifold may pump the sample gas into the diffusive light-storage cavity through an entry hole. The gas exchange manifold draws the sample gas into the diffusive light-storage cavity through an entry hole with a vacuum.

The gas exchange manifold may passively supply the sample gas to the diffusive light-storage cavity by gas diffusion.

The diffusive light-storage cavity may include a surface. The surface may include metal textured to reflect light diffusively. The surface may include one or more of gold, silver, copper, and aluminum.

The diffusive light-storage cavity may have a shape of a cube, a rectangular prism, a sphere, a serpentine chamber, or a narrow tube.

The high-efficiency LED illuminator may emit light directly into the diffusive light-storage cavity through a small hole.

The high-efficiency LED illuminator may be a mid-IR LED illuminator. The mid-IR LED illuminator may be a high-brightness mid-IR LED illuminator that has a brightness of 10 to 10,000 milliwatts per square centimeter.

The high-efficiency LED illuminator may be mounted on a circuit board that forms a surface of the diffusive light-storage cavity. The circuit board may be coated with aluminum but the high-efficiency LED illuminator may not be coated with the aluminum.

The diffusive light-storage cavity may include a sheath cavity. The gas exchange manifold may include a piston configured to enter and exit the sheath cavity.

The piston may allow the sample gas to enter the diffusive light-storage cavity by mechanically evacuating the sheath cavity.

The high-efficiency LED illuminator may emit light into the sheath cavity. The light absorption detector may receive light from the sheath cavity.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIGS. 1A and 1B illustrate a potential example container for use in sensing a sensing a target gas.

FIGS. 1C and 1D illustrate a potential spherical shaped container for use sensing a target gas.

FIG. 2 illustrates a potential example of a system for sensing a target gas.

FIG. 3 illustrates a potential example of a Lambertian gas cavity that may be used for sensing a target gas.

FIGS. 4A-4C illustrate examples of gas exchange manifolds that may be used in a Lambertian gas cavity.

FIGS. 5A and 5B illustrate examples of diffuse light-storage cavities that may be used in a Lambertian gas cavity.

FIGS. 6A and 6B illustrate an example of an illuminators and detectors integration/interfacing that may be used in a Lambertian gas cavity.

FIG. 7 illustrates an example of an immersed illuminator/detector integrated light exchange board that may be used in a Lambertian gas cavity.

FIG. 8 illustrates an example method for gas sensing.

### DETAILED DESCRIPTION

This disclosure relates generally to systems and methods for gas sensing. Typical gas-sensing methods may use multiple optical-mechanical components to focus and filter light to achieve desired results. Typical gas-sensing methods may also have a large form factor in order to create a sufficiently long optical path length. For example, background atmospheric methane measurements can require that a path length of fifty centimeters be accommodated. The disclosed systems and methods may reduce the optical-mechanical system complexity of other gas-sensing techniques while increasing the effective optical path length in a compact modular form factor.

A Lambertian gas-sensing system may enable gas sensing in a compact form factor. The Lambertian gas-sensing system may include a hollow cavity, one or more light-emitting diode (LED) illuminators, one or more light-absorption detectors, and a gas exchange manifold. The hollow cavity may mechanically integrate the gas exchange manifold, the one or more LED illuminators, and one or more light-absorption detectors (such as one or more optical detectors). The gas exchange manifold may introduce gas into the hollow cavity and the one or more LED illuminators may emit light into the hollow cavity. The one or more light-absorption detectors may receive light from the hollow cavity.

The hollow cavity may have diffusively-reflective surfaces. The hollow cavity may store isotropic light field energy from the LED illuminators using its diffusively-reflective surfaces. The diffusively-reflective surfaces may be low-absorption surfaces. The diffusively-reflective surfaces may reflect incident light such that the incident light is scattered at many angles rather than at just one angle. The diffusively-reflective surfaces may be non-specular in that they not only reflect light but also scatter the light in a broad, Lambertian pattern. Lambertian reflection may result in equal luminance when viewed from all directions lying in a half-space adjacent to the surface. Light emitted into the hollow cavity may be reflected many times while slowly being absorbed, mostly by the surfaces (walls) and small input and output ports. The hollow cavity may integrate the light emitted into the hollow cavity and spread it out evenly throughout the hollow cavity. The intensity of the light in the hollow cavity may be high because the hollow cavity may integrate the total amount of light during the time it takes the light to be absorbed. The hollow cavity may be an integrating sphere.

Hollow cavities with diffusively-reflective surfaces may help measure the total power output of a light emitter without having to carefully collect all the light emitted by the light emitter. Typically, measuring the total power of a light emitter requires the use of lenses and mirrors to focus as much emitted light as possible onto a detector. But with an integrating sphere, all light emitted by the light emitter goes into the sphere. A spherical geometry may have the best volume to surface ratio and thus the best integrating properties. Nevertheless, other arbitrary geometries, such as cubes or cylinders, can work just as well.

For gas sensing, diffusive cavities are generally used with lasers as illumination sources. Lasers are generally used because they produce high intensity light. Using a higher intensity light source means the inner surface of the integrating sphere can be less reflective. Nevertheless, using lasers requires that the inner surface be highly diffusive in order to effectively scatter light emitted from the laser.

In contrast to lasers, LEDs naturally emit light in Lambertian spatial patterns. Emitting light from LEDs into diffusive cavity radiation modes (such as the hollow cavity described above) relaxes the challenging requirement that the cavity walls have ideally diffusive reflections, which a laser illuminator requires.

For gas sensing, it may be beneficial to use mid-IR LEDs. The mid-IR spectral range is called the “molecular fingerprint” region because of the great richness of absorption features for gas and chemical targets. But using mid-IR LEDs in connection with a diffusive cavity may create certain challenges. For example, mid-IR LEDs have inherently lower efficiencies and power outputs than other wavelength LEDs. Furthermore, the absorption features of mid-IR light may limit the diffuse-reflective materials that can be used with mid-IR LEDs as compared to diffuse-reflective materials that are available for use in cavities used with light of other wavelengths. A cavity for use with mid-IR light may need to have a metallic surface to minimize absorption. Aluminum may be used because of its high reflectivity with respect to mid-IR light. Furthermore, a wealth of technology options may be available for machining and treating an aluminum surface or otherwise coating a prepared surface of another material with aluminum. In settings where aluminum may suffer corrosion and tarnishing, a less chemically reactive gold coating may be added or substituted. Other, less reflective materials, such as stainless steel, may be used. Such materials may have a shorter effective path length but gain other desirable properties, such as lower cost and better durability.

Light introduced into the hollow cavity that is not absorbed at the characteristic wavelength of the target gas species may be detected by the one or more light-absorption detectors. The one or more light-absorption detectors may include an optical detector, such as a photodiode (PD). In the alternative, the light that is absorbed by the target gas species may be indirectly detected as a heat signal by a microphone as a result of the photoacoustic (PA) effect. These light absorption signals sense the presence and concentrations of a target gas. The hollow cavity stores the emitted light from the LEDs with multiple reflections, creating longer effective optical path lengths through the absorbing target gases. The resulting longer effective optical path lengths in the hollow cavity provide a correspondingly greater sensitivity to absorption. The hollow cavity provides this greater sensitivity to absorption without requiring a large form factor. For example, the hollow cavity may have internal dimensions of only 2-4 cm but support an effective path length well over 50 cm.

A diffusive cavity (such as the hollow cavity described above) illuminated with mid-IR LEDs can serve as an optimal gas detection system. A diffusive cavity combined with mid-infrared LEDs is an excellent mechanical configuration for adding an active or passive gas sampling manifold (which allows for fabrication of a gas sensing module that integrates gas sampling); the optomechanics for light storage and absorption; illumination by mid-IR LEDs; and detection of the absorption signal by a PD, other light detector, or a photoacoustic microphone. Such a module configuration also allows multiple LEDs and detectors of varying wavelengths to be combined in the same sensor for multiple target sensing and for signal reference and calibrations.

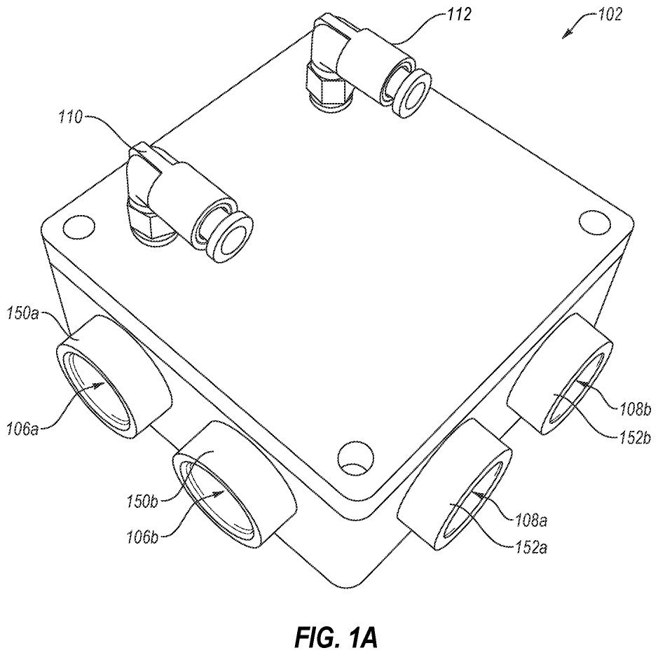

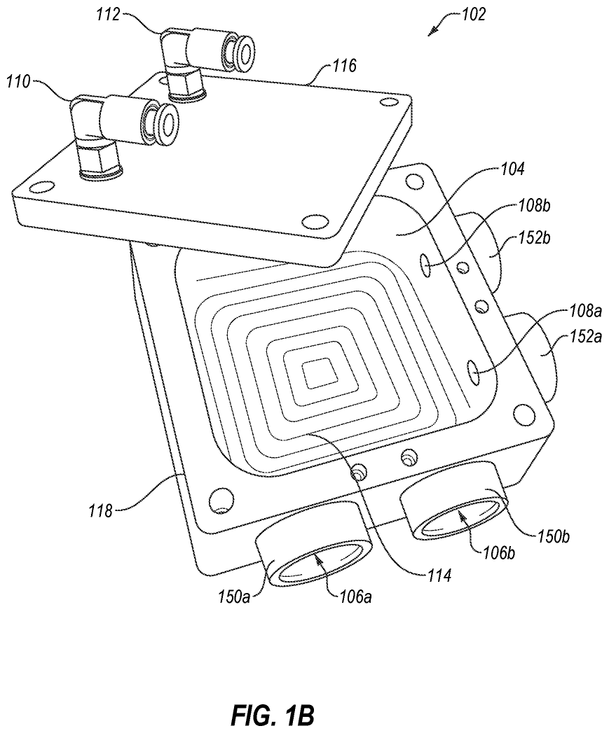

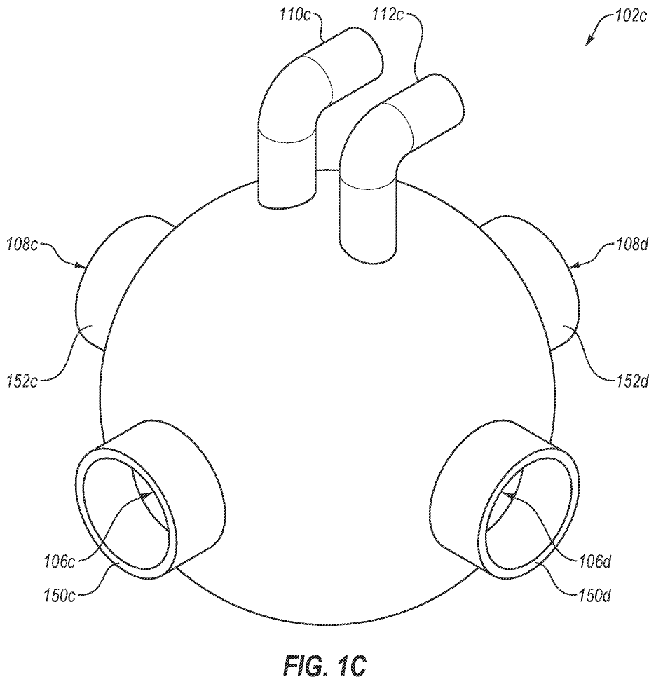

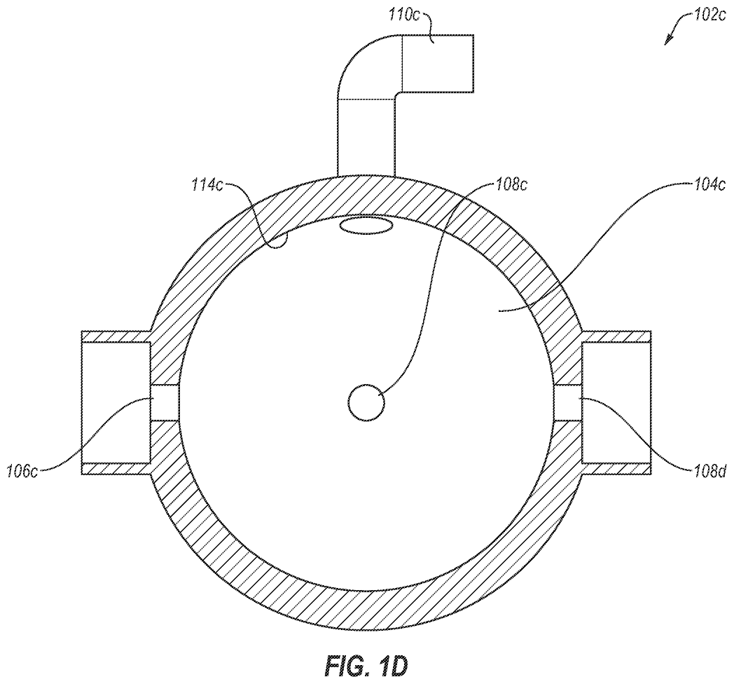

FIGS. 1A and 1B illustrate a potential example of a container 102 that may be used in a gas sensing system. The container 102 may include a hollow cavity 104, optical ports 106a, 106b, sensing ports 108a, 108b, an input port 110 that allows gas to enter the hollow cavity 104, and an output port 112 that allows the gas to leave the hollow cavity 104. The hollow cavity 104 may be a void. The hollow cavity 104 may be a space into which gas can flow. The only ways into and out of the hollow cavity 104 may be the optical ports 106a, 106b, the sensing ports 108a, 108b, the input port 110, and the output port 112. The hollow cavity 104 may be formed by more than one part. Each internal dimension (height, length, and width) of the hollow cavity 104 may be only 2-4 cm. With such dimensions, the hollow cavity 104 may support an effective path length of well over 50 cm. In FIGS. 1A and 1B, the hollow cavity 104 of the container 102 is in the form of a rectangular prism. But in other designs, a hollow cavity may have a spherical or other shape. For gas sensing, the container 102 may be used in connection with one or more LED emitters and one or more light-absorption detectors.

The container 102 may include an internal surface 114 surrounding the hollow cavity 104. The internal surface 114 may be diffusively reflective. The internal surface 114 may be reflective. The internal surface 114 may have low absorption. The internal surface 114 may scatter light in a Lambertian pattern. The internal surface 114 may integrate light emitted into the hollow cavity 104. The internal surface 114 may comprise metal. The internal surface 114 may comprise aluminum. The internal surface 114 may comprise gold. The container 102 may be constructed from a same material as the internal surface 114. In the alternative, the container 102 may be constructed from a material different from a material of the internal surface 114. For example, a container may be made from plastic but include an aluminum surface surrounding a hollow cavity.

The container 102 may include a first part 116 and a second part 118. The first part 116 may be detachable from the second part 118. The first part 116 and the second part 118 may fit together to form the hollow cavity 104. In other designs, a container may be a single unit that does not include detachable parts. In other designs, a container may include more than two parts that together form a hollow cavity.

The optical ports 106a, 106b may provide openings through which light may be emitted into the hollow cavity 104. The optical ports 106a, 106b may be small holes in the container that accommodate a light emitter but that are otherwise kept as small as possible to minimize power losses. For example, the optical ports 106a, 106b may have an area of 1 mm² or less, which may be just enough to accommodate the light emitting surface area of a light emitter, such as an LED. The optical ports 106a, 106b may have any arbitrary shape, such as a circle or a square. The optical ports 106a, 106b may be surrounded by tubes 150a, 150b on the outside of the container 102. The tubes 150a, 150b may facilitate positioning a light emitter (such as an LED) to emit light through the optical ports 106a, 106b.

The one or more LED emitters may be positioned to emit light through the optical port 106a and/or the optical port 106b into the hollow cavity 104. The one or more LED emitters may be mid-IR LEDs. The light emitted by the mid-IR LEDs may have a wavelength of 3 μm to 20 μm. The mid-IR LEDs may be high-brightness mid-IR LEDs. The mid-IR LEDs may have a brightness of 10 to 10,000 milliwatts per square centimeter. The mid-IR LEDs may have any of the properties or characteristics described in U.S. Pat. Nos. 10,374,128, 9,691,941, 9,947,827, 9,711,679, 10,529,889, which are hereby incorporated by reference in their entirety.

The sensing ports 108a, 108b may provide openings through which a light-detection device may receive light from the hollow cavity 104. The sensing ports 108a, 108b may be small holes in the container that accommodate a detector but that are otherwise kept as small as possible to minimize power losses. For example, the sensing ports 108a, 108b may have an area of 1 mm² or less, which may be just enough to accommodate a photodiode or other light sensor light collection area. The sensing ports 108a, 108b may have any arbitrary shape, such as a circle or a square. The sensing ports 108a, 108b may be surrounded by tubes 152a, 152b on the outside of the container 102. The tubes 152a, 152b may facilitate positioning a detector to receive light through the sensing ports 108a, 108b.

The one or more light-absorption detectors may be positioned to receive light through the sensing port 108a and/or the sensing port 108b. The light received through the sensing port 108a may have been emitted into the hollow cavity 104 through the optical ports 106a, 106b. The one or more light-absorption detectors may be PDs. The PDs may be mid-infrared PDs. The PDs may have any of the properties or characteristics described in U.S. Pat. Nos. 10,374,128, 9,691,941, 9,947,827, 9,711,679, 10,529,889.

The container 102 shown in FIGS. 1A and 1B includes two optical ports 106a, 106b and two sensing ports 108a, 108b. In other configurations, a container may include different numbers of optical ports and sensing ports. For example, a container may have one optical port and one sensing port. Furthermore, in other configurations, a container may have more or fewer optical ports than sensing ports. For example, a container may have two optical ports and one sensing port or two sensing ports and one optical port.

FIGS. 1C and 1D illustrate a potential example of a container 102c that may be used in a gas sensing system. The container 102c may include a hollow cavity 104c, optical ports 106c, 106d, sensing ports 108c, 108d, tubes 150c, 150d, 152c, 152d, an input port 110c that allows gas to enter the hollow cavity 104c, and an output port 112c that allows the gas to leave the hollow cavity 104c. In FIGS. 1C and 1D, the hollow cavity 104c of the container 102c is in the form of a sphere. The container 102c may include an internal surface 114c surrounding the hollow cavity 104c.

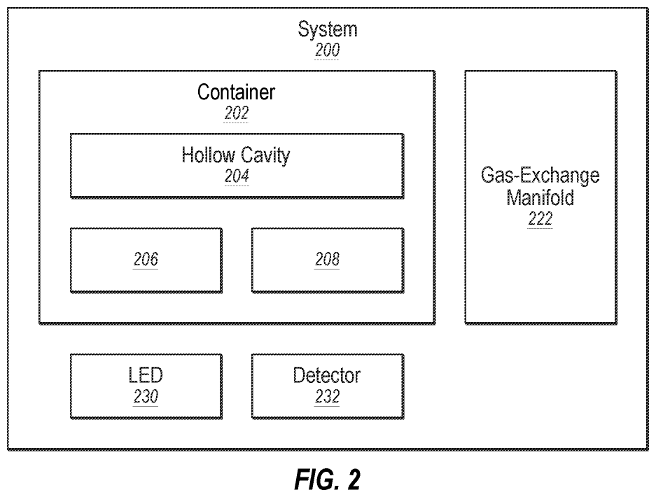

FIG. 2 illustrates one potential example of a gas sensing system 200. The system 200 may include a container 202. The system 200 may include an LED emitter 230 and a PD detector 232. The LED emitter 230 may be positioned to emit light into a hollow cavity 204 of the container 202 through an optical port 206. The PD detector 232 may be positioned to receive light from the hollow cavity 204 of the container 202 through a sensing port 208. A target gas may enter and exit the hollow cavity 204 through use of a gas exchange manifold 222. The gas exchange manifold 222 may actively pump the target gas into the hollow cavity 204 or may passively allow the target gas to enter the hollow cavity 204.

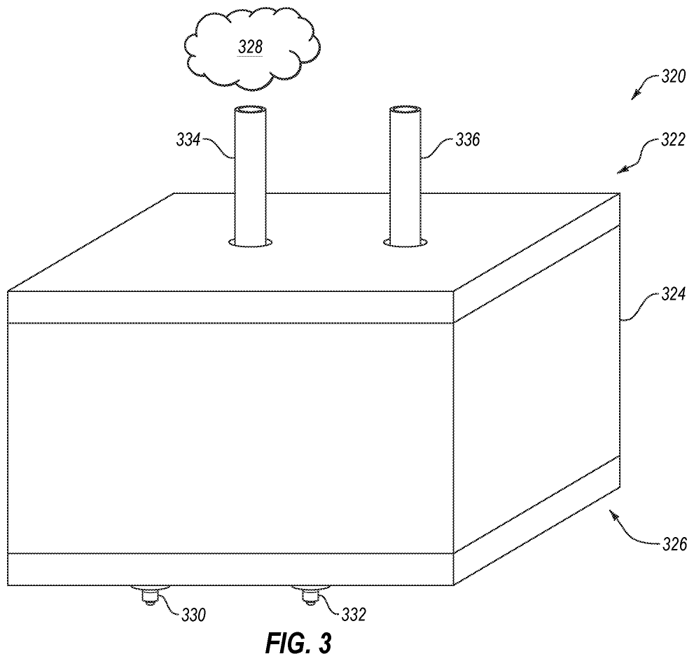

FIG. 3 illustrates a potential example of a Lambertian gas cavity (LGC) 320. The LGC 320 may include one or more subsystems. For example, the LGC 320 may include a diffuse-reflective gas manifold 322, a diffusive light storage cavity 324, and an illuminators and detectors interface/integration 326. The one or more subsystems may be integrated together.

The diffuse-reflective gas manifold 322 may allow a sample 328 to enter and exit the diffusive light storage cavity 324. The sample 328 may include a target gas. For example, the target gas may be combinations of methane, propane, or volatile organic compounds (VOCs); carbon monoxide, carbon dioxide; nitrogen oxides, NO_{R}; sulphur hexafluoride; water and many other mid-infrared absorbing gaseous or volatile compounds. The diffuse-reflective gas manifold 322 may be configured to actively supply the sample 328 by pumping the sample 328 into the diffusive light storage cavity 324 or by drawing the sample 328 into the diffusive light storage cavity 324 with a vacuum. Entry and exit holes for the sample 328 may be kept small compared to the diffusive light storage cavity 324 such that little light is lost.

The diffuse-reflective gas manifold 322 may include an input port 334 and an output port 336. The sample 328 may enter the diffusive light storage cavity 324 through the input port 334. The sample 328 may leave the diffusive light storage cavity 324 through the output port 336. The sample 328 may be actively pumped through the input port 334 into the diffusive light storage cavity 324. In the alternative, the sample 328 may be drawn into the diffusive light storage cavity 324 through the input port 334 using a vacuum. Entry and exit holes used by the input port 334 and the output port 336 may be kept small compared to the diffusive light storage cavity 324 such that little light is lost.

The diffusive light storage cavity 324 may include a hollow cavity surrounded by a surface. The surface may be diffusively reflective and have low absorption. The surface may have characteristics or features of the internal surface 114.

The illuminators and detectors interface/integration 326 may include one or more light emitters 330, one or more light detectors 332, one or more optical ports, and one or more sensing ports. The one or more light emitters 330 may emit light into the diffusive light storage cavity 324 through the one or more optical ports. The one or more light detectors 332 may receive light that has been emitted into the diffusive light storage cavity 324 through the one or more sensing ports.

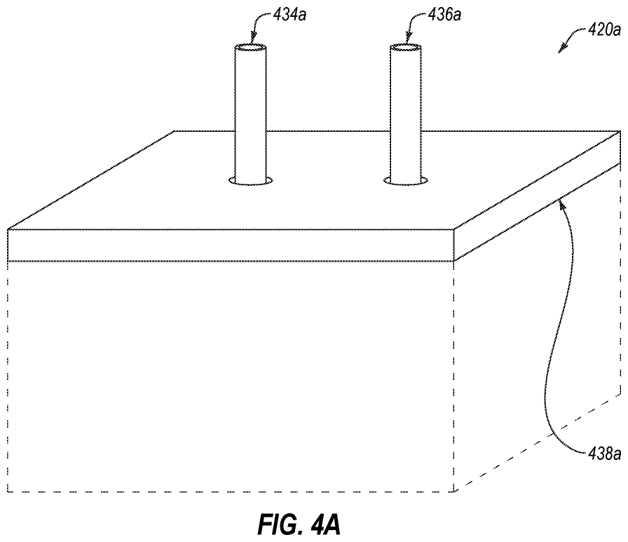

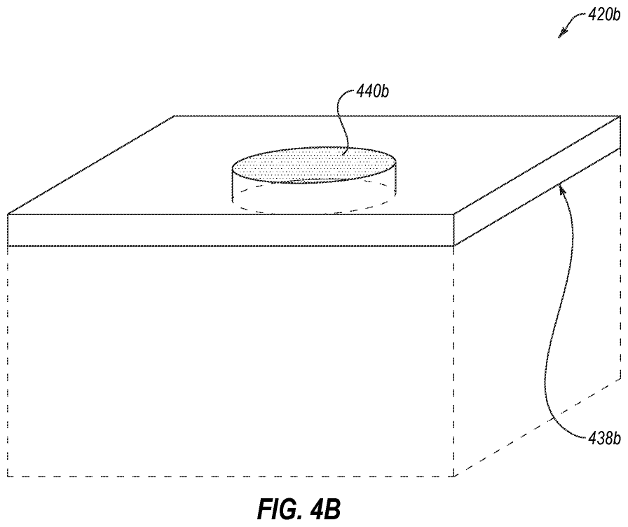

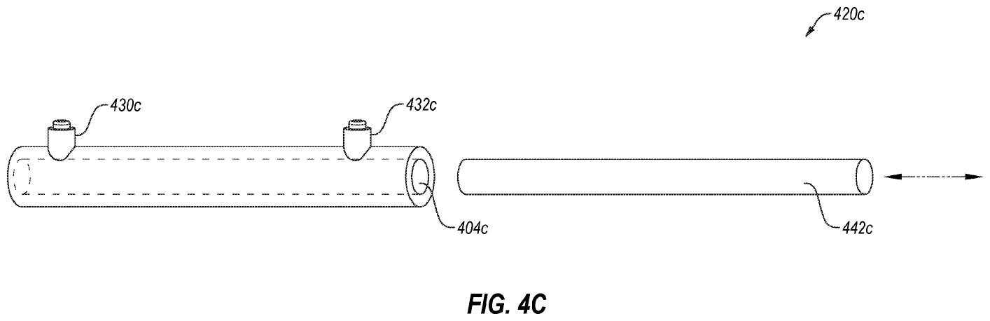

FIGS. 4A-4C illustrate examples of diffuse-reflective gas exchange manifolds that may be part of an LGC.

FIG. 4A illustrates an active exchange gas manifold 420a.

The active exchange gas manifold 420a may include an input port 434a and an output port 436a. The input port 434a may allow gases to travel from outside a diffusive light storage cavity to the inside of the diffusive light storage cavity. The output port 436a may allow gases to leave the diffusive light storage cavity. One or more gases may be actively pumped through the input port 434a into the diffusive light storage cavity. In the alternative, one or more gases may be drawn into the diffusive light storage cavity through the input port 434a using a vacuum. Entry and exit holes used by the input port 434a and the output port 436a may be kept small compared to the diffusive light storage cavity such that little light is lost.

The active exchange gas manifold 420a may include a reflective surface 438a. The reflective surface 438a may reflect light with a Lambertian or close to a Lambertian scattering pattern. The reflective surface 438a may be diffusively reflective. The reflective surface 438a may be a surface that, together with other surfaces, forms a diffusive light storage cavity.

FIG. 4B illustrates a passive gas exchange manifold 420b.

The passive gas exchange manifold 420b may include a porous light barrier 440b that allows one or more gases to pass through the passive gas exchange manifold 420b into a diffusive light storage cavity. The porous light barrier 440b may allow gas to pass into the diffusive light storage cavity by gas diffusion. The porous light barrier 440b may be a sintered barrier, a molded barrier, or a fabric barrier. The porous light barrier 440b may be designed to allow gas to pass through the porous light barrier 440b while still reflecting light within a diffusive light storage cavity. The porous light barrier 440b may be designed to reflect a maximum amount of light for a given rate of gas diffusion. The porous light barrier 440b may be designed to allow a maximum amount of gas to pass through the porous light barrier 440b for a given reflection rate. The porous light barrier 440b may be fabricated from metal. In the alternative, the porous light barrier 440b may be coated with a substance (such as aluminum) that reflects most light while still allowing the porous light barrier 440b to exchange gas. In the alternative, the porous light barrier 440b may be a designed porous structure fabricated by plastic injection molding.

The passive gas exchange manifold 420b may include a reflective surface 438b. The reflective surface 438b may reflect light with a Lambertian or close to a Lambertian scattering pattern. The reflective surface 438b may be diffusively reflective and may be aluminum or another metal. The reflective surface 438b may, together with other surfaces, form a diffusive light storage cavity.

The active and passive elements described above (such as the input port 434a, the output port 436a, and the porous light barrier 440b) may be combined, such as with an external airstream directed onto a sintered barrier.

FIG. 4C illustrates an active piston action gas exchange manifold 420c. The active piston action gas exchange manifold 420c may have a gas sample exchange take place in a piston-like fashion by mechanically evacuating a cavity. The active piston action gas exchange manifold 420c may include a sheath cavity 404c and a stylus/piston 442c. The active piston action gas exchange manifold 420c may mechanically fill and evacuate the sheath cavity 404c by inserting and removing the stylus/piston 442c. An LED 430c may emit light into the sheath cavity 404c. A PD 432c may receive light from the sheath cavity 404c. The sheath cavity 404c may be surrounded by a surface. The surface may have characteristics and features of the internal surface 114.

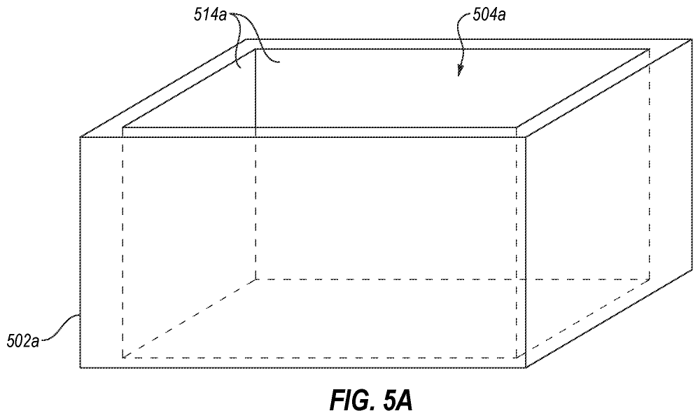

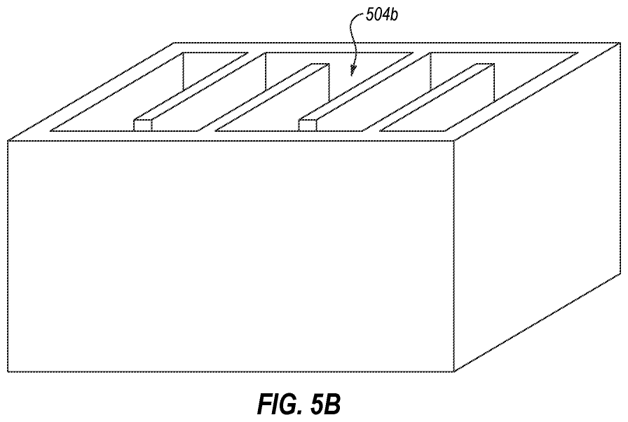

FIGS. 5A and 5B illustrate examples of a diffusive light-storage cavity 504a and a serpentine cavity 504b.

FIG. 5A illustrates the diffusive light storage cavity 504a having surfaces 514a. The diffusive light-storage cavity 504a may store isotropic light field energy from an illuminator using diffusively reflective boundaries. The diffusively reflective boundaries may include the surfaces 514, a base surface at the base of the diffusive light storage cavity 504a, and a surface of a gas exchange manifold. The diffusively reflective boundaries may also include a coated LED circuit board. The diffusively reflective boundaries may surround the diffusive light storage cavity 504a. The diffusively reflective boundaries may enclose the diffusive light storage cavity 504a. The diffusively reflective boundaries may have characteristics or features of the internal surface 114. The diffusive light storage cavity 504a shown in FIG. 5A has a shape of a rectangular prism. In other designs, a diffusive light storage cavity may have any arbitrary shape or structure. The diffusive light-storage cavity 504a may integrate a gas exchange manifold, illuminators, and optical detectors.

The surfaces 514a may be diffusively reflective. The surfaces 514a may reflect light with a Lambertian or close to a Lambertian scattering pattern. The surfaces 514a may be a metal. The metal may be structured and textured to reflect light diffusively, with a Lambertian or close to Lambertian scattering pattern. The metal may include one or more of gold, silver, copper, and aluminum. Aluminum may have reflectivity nearly as good as gold and silver but may be less expensive than gold, tarnish less than silver, and generally have more technology options for machining or coating than other metals.

A structure 502a may form the diffusive light-storage cavity 504a. The structure 502a may be constructed using aluminum. A gas manifold integrated with the structure 502a may also be constructed using aluminum. The structure 502a may be a rectangular prism. In some designs, a structure may have a shape different from a diffusive light storage cavity that it encloses. A gas manifold, illuminators, and detectors may be attached to or integrated with the structure 502a. The structure 502a and the surfaces 514a may be machined from an aluminum block or assembled from aluminum plates. A milled aluminum surface may have sufficiently diffuse reflection in its as-machined formed. A milled aluminum surface may also be further polished or brushed. If an aluminum surface cannot withstand a sampling environment and tarnishes, a gold coating may be added to create the surfaces 514a.

In the alternative, the structure 502a may be formed by injection plastic molding or with glass or ceramic. The internal walls of the structure 502 may be coated with aluminum to create the surfaces 514a. Similarly, the diffusively reflective boundaries that surround the diffusive light storage cavity 504a may be formed using an aluminum or metallic coating. A gas manifold may also be formed by injection plastic molding or with glass or ceramic.

The diffusive light-storage cavity 504a may have a size and a shape. The diffusive light-storage cavity 504a and the size and the shape of the diffusive light-storage cavity 504a may be designed to accommodate sensor geometry and a desired effective optical path length. The effective optical path length of the diffusive light-storage cavity 504a may be based on the size of the diffusive light-storage cavity 504a (which may be approximately given by its overall volume to surface area ratio (V/A)), an average surface reflectivity (ρ) of the surfaces 514a, and a total port area fraction (f). The effective path length may be chosen to match the absorption of a target gas species at an expected range of concentrations.

The diffusive light-storage cavity 504a shown in FIG. 5A is in the form of a rectangular prism. In other designs, a diffusive light-storage cavity may take other geometrical forms. For example, a diffusive light-storage cavity may be a cube. A cube has a large volume to surface area ratio, which reduces absorption losses at the surfaces. As another example, a diffusive light-storage cavity may be a long, narrow serpentine chamber (such as the serpentine cavity 504b shown in FIG. 5B). A serpentine chamber has a longer effective optical path length in a given volume but at the expense of more cavity wall absorption. As another example, a diffusive light-storage cavity may be a long narrow tube cavity (such as the sheath cavity 404c). A tube cavity may accommodate a stylus or other mechanism for piston-like gas exchange.

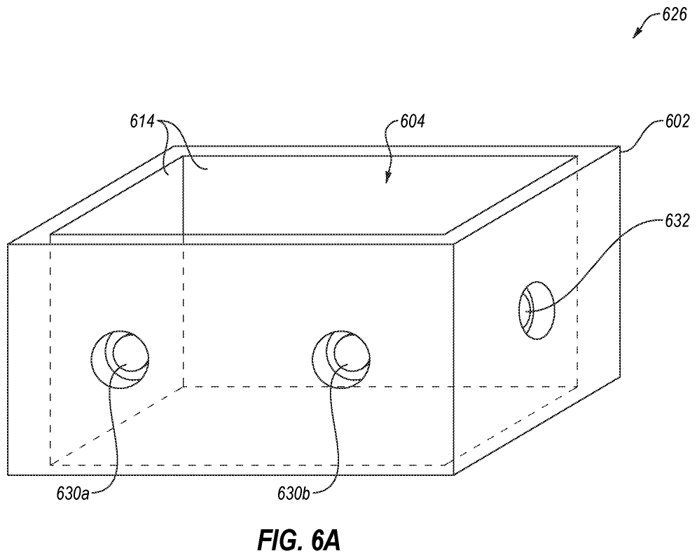

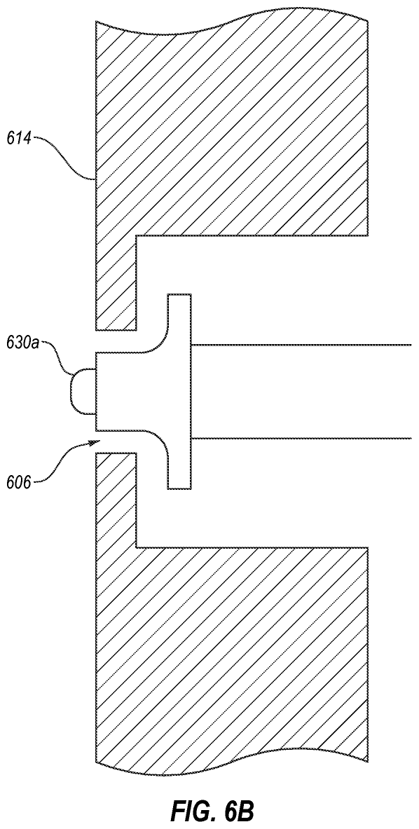

FIGS. 6A and 6B illustrate an example illuminators and detectors integration/interfacing 626. The illuminators and detectors integration/interfacing 626 may allow one or more LEDs 630a, 630b to emit light into a diffusive light-storage cavity 604 and one or more light-absorption detectors 632 to receive light from the diffusive light-storage cavity 604. The diffusive light-storage cavity 604 may be formed by a structure 602 with surfaces 614. The diffusive light-storage cavity 604 may be formed by a base surface of the structure 602. The diffusive light-storage cavity 604 may also be formed by a surface of a gas exchange manifold. The one or more LEDs 630a, 630b may be mid-IR LED illuminators. The mid-IR LED illuminators may emit illumination having a wavelength of 3 μm to 20 μm. The one or more light-absorption detectors 632 may be one or more mid-IR LED photodetectors. The one or more light-absorption detectors 632 may include a microphone directly exposed to the diffusive light-storage cavity 604 through a small hole for photoacoustic gas sensing. The one or more LEDs 630a, 630b may be high-brightness mid-IR LEDs. The one or more LEDs 630a, 630b may be high-efficiency mid-IR LEDs. A high-efficiency mid-IR LED may have an efficiency of converting electrical power to optical power of greater than 0.1 percent. The one or more LEDs 630a, 630b may be high-brightness, high-efficiency mid-IR LEDs. The one or more LEDs 630a, 630b and the one or more light-absorption detectors 632 may have any of the features or characteristics described in U.S. Pat. Nos. 10,374,128, 9,691,941, 9,947,827, 9,711,679, 10,529,889, which are incorporated in their entirety herein by reference.

The illuminators and detectors integration/interfacing 626 may be integrated with the diffusive light-storage cavity 604 and provide the one or more LEDs 630a, 630b and the one or more light-absorption detectors 632 direct exposure to the diffusive light-storage cavity 604. The illuminators and detectors integration/interfacing 626 may provide the one or more LEDs 630a, 630b and the one or more light-absorption detectors 632 direct exposure to the diffusive light-storage cavity 604 by exposing the one or more LEDs 630a, 630b and the one or more light-absorption detectors 632 to the diffusive light-storage cavity 604 through small holes. The small holes may be sized to accommodate the one or more LEDs 630a, 630b or the one or more light-absorption detectors 632 while remaining as small as possible to minimize cavity power losses. For example, a port 606 may be a hole formed in the structure 602 that provides the LED 630a direct access to the diffusive light-storage cavity 604. The port 606 may accommodate the LED 630a and allow the LED 630a to emit light into the diffusive light-storage cavity 604. The port 606 may be kept as small as possible to minimize power losses. Mid-IR LEDs may be suited to direct exposure because of their typical Lambertian emissions and because of the lower expense and optical losses of mid-IR transmissive optics and lower other focusing losses.

An optical interference filter may be used to generate greater spectral resolution than LEDs naturally provide. Collimating optics and filters may be added in front of the one or more LEDs 630a, 630b or the one or more light-absorption detectors 632. The collimating optics and filters may direct light emitted from the one or more LEDs 630a, 630b into the diffusive light-storage cavity 604 or direct light from the diffusive light-storage cavity 604 towards the one or more light-absorption detectors 632 through small apertures.

A LGC may allow for more than one target gas to be sensed at once. The diffusive light-storage cavity 604 may be illuminated by multiple LEDs for multiple sensing targets and for signal reference lights. The diffusive light storage cavity 604 may be integrated with multiple detectors of varying spectral response and with a PD detector. Each of the multiple LEDs may emit illumination of a different wavelength. The wavelength of illumination emitted by each of the multiple LEDs may be based on the light absorption characteristics of each of multiple target gases. For example, a first LED may emit illumination having a wavelength of 3.3 μm based on the absorption characteristics of methane, and a second LED may emit illumination having a wavelength of 4.3 μm based on the absorption characteristics of carbon dioxide. Another example may involve a first LED having a wavelength of 3.6 μm based on the absorption characteristics of formaldehyde and other VOCs and a second LED having a wavelength of 4.7 μm based on the absorption characteristics of carbon monoxide. Similarly, the spectral response of each of the multiple detectors may be based on the absorption characteristics of each of the multiple target gases.

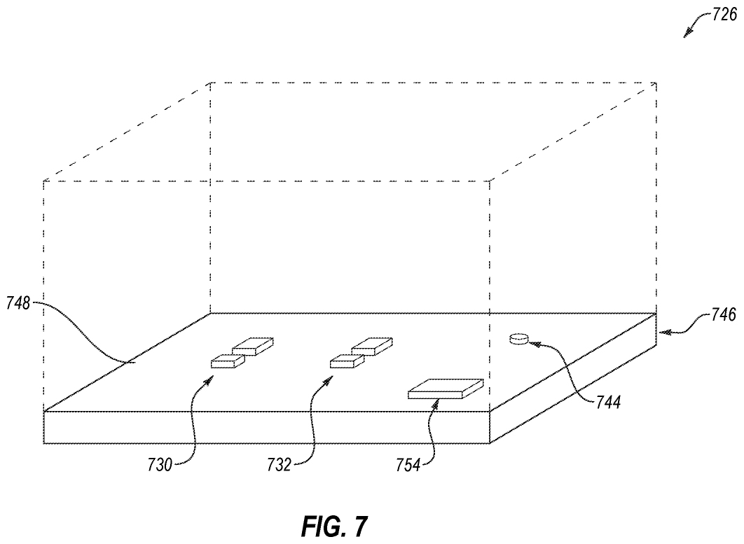

FIG. 7 illustrates an example immersed illuminator/detector integrated light exchange board 726. The immersed illuminator/detector integrated light exchange board 726 may include an LED printed circuit board 746. The LED printed circuit board 746 may include LED chips 730, light-absorption detector chips 732, a microphone 744, and other electrical components (such as microphone control chips) 754. The immersed illuminator/detector integrated light exchange board 726 may provide the LED chips 730 and the light-absorption detector chips 732 direct access to a diffusive light-storage cavity. The immersed illuminator/detector integrated light exchange board 726 may help form the diffusive light-storage cavity.

The LED printed circuit board 746 may include a coating 748. The coating 748 may be aluminum or another metal. The coating 748 may cover the LED printed circuit board 746 on a side facing a diffusive light storage cavity. The coating 748 may not cover the LED chips 730 or the light-absorption detector chips 732. The coating 748 may maximize reflectivity of the diffusive light storage cavity. A thin insulating layer may be placed between the LED printed circuit board 746 and the coating 748 to protect the LED printed circuit board 746 and its components. The coating 748 may, together with other surfaces, form a diffusive light-storage cavity.

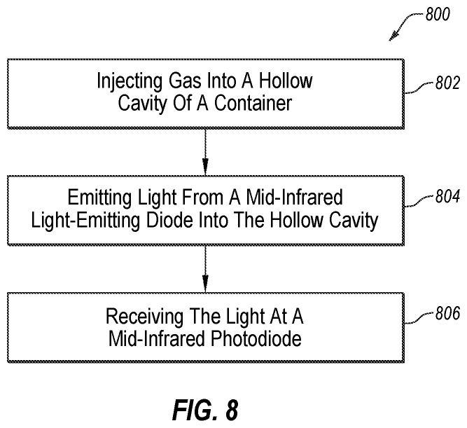

FIG. 8 illustrates an example method 800 for gas sensing.

The method 800 may include injecting 802 gas into a hollow cavity of a container. The container may include one or more optical ports and one or more sensing ports. The hollow cavity may be a rectangular prism. The hollow cavity may be spherical. The container may include an internal surface surrounding the hollow cavity. The internal surface may be diffusively reflective. The internal surface may be reflective, may have low absorption, and may scatter light in a Lambertian pattern. The internal surface may be aluminum. The container may be formed from plastic, and the internal surface surrounding the hollow cavity may be aluminum. The container may have a first part and a second part. The first part may be detachable from the second part. The first part and the second part may fit together to form the hollow cavity.

The method 800 may include emitting 804 light from a mid-infrared light-emitting diode (LED) into the hollow cavity. The mid-infrared LED may emit the light into the hollow cavity through the one or more optical ports. The light emitted from the mid-infrared LED may have a wavelength of 3 μm to 20 μm. The mid-infrared LED may be a high-brightness mid-infrared LED.

The method 800 may include receiving 806 the light at a mid-infrared photodiode. The mid-infrared photodiode may receive the light from the hollow cavity through the one or more sensing ports.

The method 800 may further include allowing at least a portion of the gas to leave the hollow cavity.

1. A system for gas sensing, the system comprising:
a container comprising a hollow cavity, an optical port, a sensing port, an input port that allows gas to be injected into the hollow cavity, and an output port that allows the gas to leave the hollow cavity;
a mid-infrared light-emitting diode (LED) configured to emit light into the hollow cavity through the optical port; and
a mid-infrared photodiode configured to receive the light from the hollow cavity through the sensing port.

2. The system of claim 1, wherein the light emitted by the mid-infrared LED has a wavelength of 3 μm to 20 μm.

3. The system of claim 2, wherein the mid-infrared LED is a high-brightness mid-infrared LED.

4. The system of claim 3, wherein the hollow cavity is a rectangular prism.

5. The system of claim 4, wherein the hollow cavity is spherical.

6. The system of claim 5, wherein the container comprises an internal surface surrounding the hollow cavity and the internal surface is diffusively reflective.

7. The system of claim 6, wherein the container comprises an internal surface surrounding the hollow cavity and the internal surface is reflective, has low absorption, and scatters the light in a Lambertian pattern.

8. The system of claim 7, wherein the container comprises an internal surface surrounding the hollow cavity and the internal surface comprises aluminum.

9. The system of claim 8, wherein the container comprises plastic and an internal surface surrounding the hollow cavity, the internal surface comprising aluminum.

10. The system of claim 9, wherein the container comprises a first part and a second part, the first part being detachable from the second part, wherein the first part and the second part fit together to form the hollow cavity.

11. A method for gas sensing, the method comprising:
injecting gas into a hollow cavity of a container, wherein the container comprises an optical port and a sensing port;
emitting light from a mid-infrared light-emitting diode (LED) into the hollow cavity through the optical port; and
receiving, through the sensing port, the light at a mid-infrared photodiode.

12. The method of claim 11, the method further comprising:
allowing at least a portion of the gas to leave the hollow cavity.

13. The method of claim 12, wherein the method further comprises emitting light from the mid-infrared LED has at a wavelength of 3 μm to 20 μm.

14. The method of claim 13, wherein the method further comprises emitting light from the mid-infrared LED using a high-brightness mid-infrared LED.

15. The method of claim 14, wherein the method further comprises using a rectangular prism as the hollow cavity.

16. The method of claim 14, wherein the method further comprises using a spherical hollow cavity.

17. The method of claim 14, wherein the method further comprises surrounding the hollow cavity with an internal surface that is diffusively reflective.

18. The method of claim 17, wherein the method further comprises making the internal surface surrounding the hollow cavity reflective, having low absorption, and scattering the light in a Lambertian pattern.

19. The method of claim 18, wherein the method further comprises surrounding the hollow cavity and the internal surface with aluminum.

20. (canceled)

21. The method of claim 19, wherein the method further container comprises making a first part and a second part, the first part being detachable from the second part, wherein the first part and the second part fit together to form the hollow cavity.

22-39. (canceled)
